(19) United States
(12) Patent Application Publication
(10) Pub. No.: US 2022/0120721 A1
(43) Pub. Date: Apr. 21, 2022

(54) KIT AND METHOD FOR DETERMINATION OF FENTANYL DRUGS IN BIOLOGICAL SAMPLES

(71) Applicant: Ningbo Municipal Center for Disease Control and Prevention, Ningbo City (CN)

(72) Inventors: Jian Zhou, Ningbo City (CN); Micong Jin, Ningbo City (CN); Xiaohong Chen, Ningbo City (CN); Chenlu Wang, Ningbo City (CN); Jiancheng Yu, Ningbo City (CN)

(21) Appl. No.: 17/482,889

(22) Filed: Sep. 23, 2021

Related U.S. Application Data
(63) Continuation of application No. PCT/CN2021/074594, filed on Feb. 1, 2021.

(30) Foreign Application Priority Data
Oct. 16, 2020 (CN) 202011106966.5

(51) Int. Cl. G01N 30/72; G01N 30/06

(52) U.S. Cl. CPC G01N 30/7233; G01N 2030/027; G01N 2430/00; G01N 30/06

(57) ABSTRACT

A kit and a method for determination of fentanyl drugs in biological samples are provided, belonging to the field of biotechnology. The method includes: a sample is transferred into a centrifuge tube containing acetonitrile in advance for shaking, extraction and centrifugation; a supernatant is drawn into a purification extraction column by pulling a plunger upwards and fully contacted with absorbents to quickly complete a preliminary purification; the plunger is pulled upwards continuously to absorb a certain volume of air, then a filter is installed at the bottom of the purification extraction column and the plunger is pushed downwards to make a sample extractant comes into contact with mixed absorbents. The filtrate is collected and subjected to analysis on liquid chromatography-tandem mass spectrometry. Compared with existing approaches, the proposed methodology is simpler, faster, more efficient, minimizes the impact caused by insufficient professional experience, thus greatly improves accuracy and precision results.

### TECHNICAL FIELD

The disclosure belongs to the field of biotechnology, in particular to a kit and a method for determination of fentanyl drugs in biological samples.

### BACKGROUND

Fentanyl, a synthetic opioid drug, was first synthesized by Belgian scientist Paul Janseen in 1960 and sold as an analgesic, whose analgesic effect is 80 times that of morphine. Fentanyl (and its analogues) binds to opioid receptor and has high affinity, high fat solubility and strong intrinsic activity. These are not only the important pharmacological characteristics of fentanyl, but also the primary cause of its fatal adverse reactions (respiratory depression and even death), which is manifested in that it has strong analgesic effect and high abuse potential. Fentanyl drugs can quickly penetrate the cell membrane to the blood-brain barrier and enter the brain to form a blood drug peak for a short time, and is prone to form tolerance and drug dependence. Fentanyl can be absorbed through the skin and mucous membranes, so poisoning of this kind of substance occurs not only among abusers, but also among staff members who handle or are exposed to fentanyl drugs without protective measures.

Fentanyl drug is widely employed in clinic due to its powerful analgesic effect, but meanwhile, the phenomenon of non-medicinal use and abuse of fentanyl have also appeared. Recently, fentanyl has been detected in international parcels as components of unidentified powder, tablets and capsules and also appeared on the market in some countries. Thus, the illicit drugs containing fentanyl analogues are possible to be disguised as health products via e-commerce. At present, there are various analytical methods that can be used for the determination of fentanyl and its derivatives in biological samples, such as immunoassay, gas chromatography-mass spectrometry (GC-MS), liquid chromatography-mass spectrometry (UHPLC-MS/MS), etc. Immunoassay is a method characterized by the specific binding of antigen (target) and antibody, but different immunoassay methods have limitations on the cross-reaction of fentanyl analogues and some cross-reactions remain unknown. The MS coupled to either UHPLC or GC is acknowledgedly recognized as the powerful tool in clinical applications for the analysis of drug analytes. GC-MS was the gold standard for the previous decade, but it lacks sensitivity to detect trace-level concentration of analytes and moreover requires cumbersome derivatization of the non-volatile, polar or thermally unstable compounds. In this context, UHPLC-MS/MS method is a robust alternative for the determination of fentanyl and its analogues in the complex matrix, and is superior to GC/MS in terms of scope of application, sensitivity, analysis speed, etc. The sample preparation strategies remain the primary challenge and are always required prior to LC-MS analysis. In this case, very few studies have been published on the development and validation of LC-MS/MS methods for the identification and quantification of several molecules belonging to the fentanyl's family. Therefore, a reliable, stable and efficient method for the determination of fentanyl drugs is urgently needed and applied to solve practical problems.

### SUMMARY

In view of the problems existing in the previous studies, the disclosure aims to provide a kit and a method for the determination of fentanyl drugs in biological samples.

In order to achieve the above objectives, the disclosure provides the following scheme:

A method for determination of a fentanyl drug(s) in a biological sample, exemplarily employing ultra-high performance liquid chromatography tandem mass spectrometry (UHPLC-MS/MS) for determining a content(s) of the fentanyl drug(s) in the biological sample, and specifically including:

step (1) sample pretreatment: shaking/vibrating and centrifuging the sample sequentially, and then removing co-extraction impurities in an extract of the centrifuged sample by a purification extraction tube assembly for fentanyl drugs to thereby obtain a target solution;

step (2) LC-MS/MS based analysis on the target solution: using 0.1% by volume of formic acid-aqueous solution and 0.1% by volume of formic acid-acetonitrile as mobile phases for liquid chromatography analysis, and using a multiple-reaction monitoring (MRM) mode under positive c (ESI) for mass spectrometry analysis.

Further, the fentanyl drug may be one of: acetylfentanyl, isobutyrylfentanyl, acrylfentanyl, ocfentanyl, fentanyl, valerylfentanyl and furanylfentanyl.

Further, the purification extraction tube assembly may be composed of mixed purifying agents, a blank solid-phase extraction column, two sieve plates, a polytetrafluoroethylene filter membrane, and a syringe plunger.

Further, the applicative sample types may include whole blood, saliva, and urine.

Further, the specific components and dosages of the mixed purifying agents mentioned in Step (1) are as follows: 27 mg of C₁₈, 29 mg of EMR (EMR-Lipid), 143 mg of NH₂ (aminopropyl), 100 mg of PSA (primary secondary amine), 100 mg of alkaline diatomite and 100 mg of basic alumina.

Further, the specific components and dosages of the mixed purifying agents may be ascertained through chemometrics techniques.

Further, the chemometrics techniques may include Plackett-Burman design and central composite design based on response surface methodology, used for screening and further optimization, respectively.

Further, the filter membrane may be a 0.22 μm hydrophilic PTFE (polytetrafluoroethylene) millipore/microporous filtration membrane.

Further, the shaking and centrifuging the sample sequentially in the step (1) is: 2 times the sample volume of acetonitrile is added with the sample together for the precipitation of proteins. The mixture is vortexed/shaken vigorously for 5 min and then centrifuged at 15000 rpm for 10 min (4° C.). The supernatant obtained after the centrifugation is aspirated into the purification extraction tube assembly to make it in full contact with the mixed purifying agents, and then pushed out again after installing the filter membrane in the front of the solid phase extraction column.

Further, conditions for the liquid chromatography analysis in the step (2) may be as follows: mobile phase A: 0.1% formic acid solution (V/V), mobile phase B: 0.1% formic acid-acetonitrile (V/V), separation/chromatography column: Waters BEH C₁₈ column (100 mm×2.1 mm, 1.7 μm), flow rate: 300 μL/min, and injection volume: 10 μL.

Conditions for the mass spectrometry analysis may be that: electrospray ionization in positive mode is performed in multiple-reaction monitoring (MRM) conditions, nitrogen is used as curtain gas and collision gas at 20.0 psi and 7.0 psi respectively, declustering voltage: 120 V, ionspray voltage maintained at 4.5 kV, and source cone temperature: 500° C.

In another embodiment of the disclosure, a kit for determination of a fentanyl drug(s) in a biological sample according to any one of the above methods is provided and includes: a purification extraction tube assembly, polypropylene centrifuge tubes prefilled with acetonitrile, standard working solutions, quality-control samples, and several disposable consumables

The disclosure provides a kit for the preparation of fentanyl drugs in biological samples, which may include a purification extraction tube assembly, polypropylene centrifuge tubes that prefilled with acetonitrile, standard working solutions, quality control samples and several disposable consumables like centrifuge tubes, nitrile gloves and syringes.

The disclosure discloses the following technical effects:

1. The disclosure develops a kit and a method for rapid preparation of 7 kinds of fentanyl drugs in plasma, saliva and urine, in which a novel type of purification extraction tube assembly is treated as the main body. The purification extraction tube assembly works analogous to the dispersive-solid phase extraction technique, several purifying agents are pre-proportional mixed and fixed with sieve plates. The operational motion of pretreatment is similar to the action of sucking and injecting liquid using a syringe. More specifically, with the aid of a plunger, the sample extractant can be made to have full contact with the packaged agents twice within 1 min, thus completing the purification quickly and efficiently. Compared with the traditional purification methods, the operation mode provided by this disclosure is more simple and more efficient, no other extra steps (like concentration, re-dissolution, etc.) are required. The above characteristics allow to avoid the issue of inferior precision and accuracy caused by the non-professional technical personnels.

2. Chemometrics techniques, including Plackett-Burman design and response surface methodology, are applied to screen out the critical factors, to identify the interaction effects of the significant variables and to reach the optimal conditions. More detailedly, the purpose of Plackett-Burman design is to make sure that the factors being further optimized do indeed significantly contribute to the responses and thus narrowing the investigation range of candidate variables. After the critical factors have been ascertained, central composite design involves estimating the coefficients by fitting the experimental data to the response functions, checking its goodness-of-fit, identifying important interactions and searching the theoretical optimal conditions. Plackett-Burman design and central composite design can ensure that the designed experiments provide the maximum amount of relevant information with a minimum number of runs.

3. The proposed purification extraction tube assembly can effectively reduce the co-extracted interferences (also referred to as co-extraction impurities) and thus keep the matrix effects of analytes within acceptable ranges. The mixed purifying agents are proved to have good uniformity and stability, meanwhile, the results of accuracy, precision and sensitivity of this method are satisfactory.

4. The amounts of reagents used in the kit has been ascertained in advance and prefilled in tubes, and only a handheld centrifuge is needed to complete the whole sample pretreatment process. The designed kit is very suitable for on-site sampling and essential clean-up. In the follow-up, it can be linked up with portable or desktop mass spectrometry equipment to quickly complete the quantitative analysis on 7 fentanyl drugs in the samples, which is a unique predominance comparing with the prevalent methods.

### BRIEF DESCRIPTION OF DRAWINGS

FIG. 1 is a diagrammatic sketch of an operation flow of samples employing a purification extraction tube assembly.

FIG. 2 shows MRM spectra of 7 kinds of fentanyl drugs at 5.0 ng/mL.

FIG. 3 is a schematic diagram of the design of the purification extraction tube assembly.

FIGS. 4A-4B represent total ion chromatograms for purification effect comparison, where FIG. 4A is a total ion chromatogram of the sample after being purified using the purification extraction tube assembly, and FIG. 4B is a total ion chromatogram of the sample without purification.

FIGS. 5A-5C are diagrams associated with three-level spiking tests in three investigated matrices.

### DETAILED DESCRIPTION OF EMBODIMENTS

The embodiments of the disclosure are further illustrated in the drawings below, which shall not be considered as a limitation to the disclosure but shall be construed as a more detailed description of certain aspects, characteristics and implementation schemes of the disclosure.

It shall be understood that the terminology described herein is for the purpose of describing particular embodiments only and is not intended to be the limitation to the disclosure. In addition, for the numerical range in the disclosure, it should be understood that each intermediate value between the upper limit and the lower limit of the range is also specifically disclosed. Each smaller range between any stated values or intermediate values within the stated range and any other stated value or every small range between intermediate values within the stated range are also included in the disclosure. The upper and lower limits of these smaller ranges may be independently included within or excluded from the range.

Unless otherwise indicated, all technical and scientific terms used herein have the same meaning as that commonly understood by those skilled in the art in the field of the disclosure. Although the disclosure describes only preferred methods and materials, any methods and materials similar or equivalent to those described herein may be used in the implementation or testing of the disclosure. All documents mentioned in this specification are incorporated by reference for the purpose of disclosing and describing methods and/or materials related to the documents. In case of conflict with any incorporated document, the contents of this specification shall prevail.

Various modifications and variations can be made in the specific embodiments of the present specification without departing from the scope or spirit of the disclosure, which is apparent to those skilled in the art. Other embodiments derived from the disclosure will be apparent to those skilled in the art. The specification and embodiments of the present application are only exemplary.

As used herein, “comprising”, “including”, “having”, “containing” and the like are all open terms, which means including but not limited to.

The reagents and materials and experimental instruments involved in the disclosure are as follows:

A. Reagents and Materials

Standard solution of acetylfentanyl, isobutylfentanyl, propylfentanyl, ocfentanyl, fentanyl, valeryfentanyl and furanylfentanyl (100 micrograms per milliliter (μg/mL) in methanol), Sigma-Aldrich (Shanghai) Trading Co., Ltd.; methanol (CH₃OH, chromatographic grade), acetonitrile (C₂H₃N, chromatographic grade), Fisher Scientific (China) Co., Ltd.; QuEChERS purifying agent (including C₁₈ adsorbent, PSA (primary secondary amine adsorbent), EMR (Bond Elut EMR-Lipid), alkaline diatomite, neutral diatomite, florisil, acidic alumina, neutral alumina, alkaline alumina, NH₂ (aminopropyl), GCB (graphitized carbon black), Phenomenex & Agela Technology Co., Ltd., Agilent Technology (China) Co., Ltd.; empty solid phase extraction column (6 mL), solid phase extraction micropore filters ( 1/16, 10 micrometers (μm)), Phenomenex & Agela Technology Co., Ltd.; 0.22 μm hydrophilic polytetrafluoroethylene filter, Anpel Laboratory Technologies (Shanghai) Co., Ltd.; disposal syringes (1 mL), Zhejiang Gongdong Medical Technology Co., Ltd.; Waters BEH C₁₈ column (100 millimeters (mm)×2.1 mm, 1.7 μm), Waters Technology (Shanghai) Co., Ltd.; other reagents and consumables are purchased from local suppliers. In summary, the kit contains a number of purification extraction tube assemblies and plungers, polypropylene centrifuge tubes prefilled with 2 mL of acetonitrile, standard working solutions, quality control samples, disposable syringes, vacuum blood collection tubes, several spare centrifuge tubes and gloves.

B. Experimental Instruments

ExionLC Liquid Chromatography, Shimadzu, Japan; Sciex Q-Trap 6500 plus mass spectrometry, Sciex Corporation, USA; Milli-Q Ultrapure Water purifier, Millipore Pore, USA.

### Embodiment 1

Determine the Concentrations of Fentanyl Drugs in Biological Samples

1.1 Preparation of Standard Stock Solution

The mixed stock solution of fentanyl drugs is prepared individually in acetonitrile to yield a concentration of 10.0 μg/mL and stored at −20° C. for 12 months.

1.2 Preparation of Mixed Standard Working Solution

The mixed working solution is prepared by serially diluting the stock solution to yield a final concentration of 1.0 μg/mL and stored at −20° C. for 1 months.

1.3 Chromatographic Conditions

Chromatographic separation is carried out using a gradient elution with eluent A being 0.1% formic acid aqueous solution (V/V) and eluent B being 0.1% formic acid-acetonitrile (V/V) on a Waters BEH C₁₈ column (2.1 mm×100 mm, 1.7 μm) with column temperature at 40° C., the flow rate of 0.3 milliliters per minute (mL/min), and the sample volume of 10 microliters (μL). The liquid gradient condition is shown in Table 1.

TABLE 1
Detailed gradient program

| | Mobile | Mobile |
|---|---|---|
| Time/min | phase A | phase B |
| 0.00~2.00 | 70%~70% | 30%~30% |
| 2.00~2.10 | 70%~50% | 30%~50% |
| 2.10~5.50 | 50%~50% | 50%~50% |
| 5.50~5.60 | 50%~2% | 50%~98% |
| 5.60~7.00 | 2%~2% | 98%~98% |
| 7.00~7.10 | 2%~70% | 98%~30% |
| 7.10~9.00 | 70%~70% | 30%~30% |

1.4 Mass Spectrometry Parameters

Ion source: electrospray ionization; positive mode; ionspray voltage: 5.0 kilovolts (kV), source temperature: 500° C., curtain gas: 20 pounds per square inch (psi); collision gas: 7 psi. Acquisition is performed in multiple-reaction-monitoring mode, and the specific MS/MS parameters such as the selection of precursor and product ions are summarized in Table 2.

TABLE 2
Multi-reaction monitoring conditions for fentanyl drugs

| | Precursor | Declustering | Quantitative | Collision | Qualitative | Collision |
|---|---|---|---|---|---|---|
| Compounds | ion (m/z) | potential (V) | ion (m/z) | voltage (eV) | ion (m/z) | voltage (eV) |
| Acetylfentanyl | 323.3 | 80 | 188.0 | 30 | 105.0 | 50 |
| Isobutyrylfentanyl | 351.2 | 80 | 188.0 | 30 | 105.0 | 50 |
| Acryloylfentanyl | 335.2 | 80 | 188.0 | 30 | 105.0 | 50 |
| Ocfentanyl | 371.2 | 80 | 188.0 | 30 | 105.0 | 50 |
| Valerylfentanyl | 365.2 | 80 | 188.0 | 30 | 105.0 | 50 |
| Furanylfentanyl | 375.4 | 80 | 188.0 | 30 | 105.0 | 50 |
| Fentanyl | 337.4 | 80 | 188.0 | 30 | 105.0 | 50 |

1.5. Sample Extraction

Whole blood sample: More than 3 mL of whole blood sample is collected and centrifuged at 2000 rpm for 5 min, to obtain the blood plasma. 0.5 mL of the plasma is accurately transferred to the centrifuge tube, in which 2 mL of acetonitrile has been prefilled. The excess sample is packed into a 50-mL centrifugal tube and preserved at −20° C. The mixture of plasma and acetonitrile is vortexed vigorously through a rotary-shaking stirrer and then centrifuged at 15000 revolutions per minute (rpm) and 4° C. for 10 min.

Saliva and urine samples: 1.0 mL of saliva or urine samples is accurately transferred to the centrifuge tube (attention should be paid to minimize the air bubbles), in which 2 mL of acetonitrile has been prefilled. The mixture of sample and acetonitrile is vortexed vigorously through a rotary-shaking stirrer and then centrifuged at 15000 rpm and 4° C. for 10 min.

1.6 Purification Procedure

At first, the sealing plug and silicon rubber plug are removed, and then the plunger is installed into the solid phase extraction column. The head of the solid phase extraction column is extended below the surface of the extractant, and then the plunger is slowly pulled up.

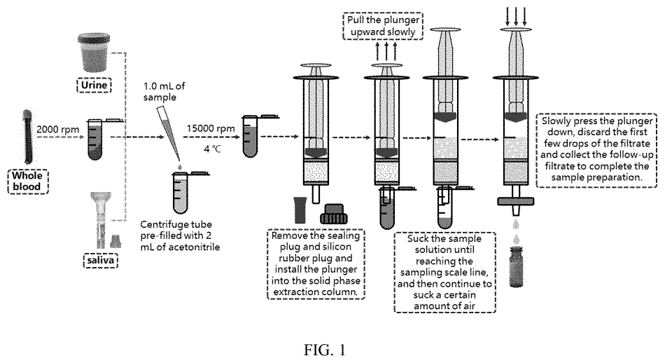

The operation procedure diagram is shown in FIG. 1, namely, the pre-treatment schematic diagram of the purification unit. The upper end seal plug and the lower end silicone sleeve of the extraction purification pipe are removed, and the syringe plunger is put into the solid phase extraction column and pushed to the bottom. The bottom end of the solid phase extraction column is extended below the liquid level of the extracting solution, and the plunger is pulled upward slowly. It should be noted that the entire sampling process should be maintained for at least 0.5 min, in order to make the sample solution fully contact with the purifying agents. After enough extractant had been sucked into the purification extraction tube assembly, the plunger is still pulled upward continuously to allow certain volume of air to enter the tube. Then, a 0.22 μm hydrophilic PTFE millipore filter is installed at the bottom of the solid phase extraction column. The plunger is pressed downward slowly and the remaining filtrate is collected after the first 3-4 drops of liquid being discarded. The purified filtrate is one-to-one diluted using 0.1% formic acid prior to UHPLC-MS/MS sample analysis.

### Embodiment 2

Optimization of UHPLC-MS/MS Conditions

The pure standard solutions are infused directly into the mass spectrometer in full-scan mode to get the accurate precursor parameters. The ionization efficiencies of these drugs in electrospray ionization are significantly related to the pH condition of mobile phase. More specifically, protonated products, i.e., [M+H]⁺, are normally obtained under the positive mode using 0.1% formic acid as the aqueous phase.

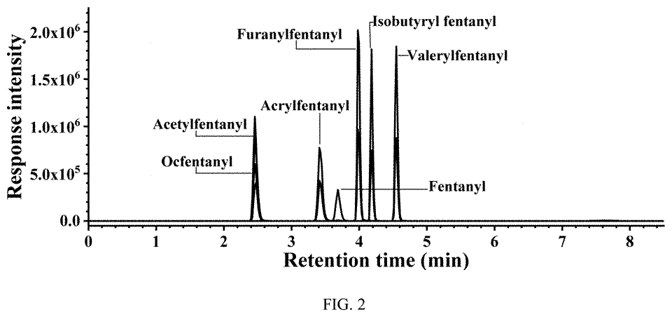

On the other hand, the fentanyl drugs have medium polarity, and the ionization efficiencies of these drugs in electrospray ionization is significantly related to the pH condition of mobile phase. When no formic acid or 0.1% ammonia is added to the mobile phase, the ionization efficiencies of all target drugs are extremely low, but after 0.1% formic acid is added to the aqueous phase and organic phase, the response intensity of the drugs increased significantly. Acetonitrile and methanol are considered as the potential organic phases and assessed in terms of column backpressure, separation effect and analysis time. As a result, compared with methanol, acetonitrile generated relatively lower pressure, facilitated the elution of drugs. Finally, 0.1% formic acid-aqueous solution and 0.1% formic acid-acetonitrile are selected as the mobile phases, and the typical MRM spectrum of standard solution (5.0 nanograms per milliliter (ng/mL)) is shown in FIG. 2.

### Embodiment 3

Optimization Strategy for Mixed Purifying Agents

1. Evaluation of Sorbents

The frequently-used QuEChERS sorbents are involved, including C₁₈ adsorbent, PSA (primary secondary amine adsorbent), EMR (Bond Elut EMR-Lipid), alkaline diatomite, neutral diatomite, florisil, acidic alumina, neutral alumina, alkaline alumina, NH₂ (aminopropyl), GCB (graphitized carbon black). Although QuEChERS method has been proven to be suitable for multi-component analysis, this technique still suffers from the problem of analyte loss when using most absorbents. The experiment conditions are as follows: 1.0 mL of standard solution (5.0 ng/mL) is mixed with 2.0 mL of acetonitrile, and then transferred to a dispersive tube containing 300 mg of each test sorbent. The mixture is vortexed for 10 min and then centrifuged (8500 rpm, 3 min), 200 μL of supernatant is transferred and diluted with 800 μL of pure water prior to analysis (results shown in Table 3).

TABLE 3
The Recovery Results of Fentanyl Drugs Using Different QuEChERS Sorbents

| Adsorbent | Acetylfentanyl | Isobutyrylfentanyl | Acrylfentanyl | Ocfentanyl | Valerylfentanyl | Furanylfentanyl | Fentanyl |
|---|---|---|---|---|---|---|---|
| C₁₈ | 67.2 | 60.5 | 65.4 | 75.3 | 50.4 | 66.8 | 67.5 |
| PSA | 90.8 | 90.0 | 88.0 | 90.4 | 84.5 | 80.6 | 84.3 |
| EMR | 68.8 | 72.9 | 68.5 | 78.7 | 65.6 | 57.3 | 66.4 |
| Alkaline diatomite | 93.5 | 94.5 | 89.1 | 94.7 | 81.1 | 90.7 | 90.4 |
| Neutral diatomite | 88.8 | 92.2 | 91.2 | 90.3 | 75.3 | 85.0 | 94.2 |
| Florisil | 0.0 | 0.0 | 0.0 | 0.0 | 0.0 | 0.0 | 0.0 |
| Acidic alumina | 0.4 | 2.1 | 1.3 | 0.7 | 1.4 | 0.8 | 0.7 |
| Neutral alumina | 66.9 | 81.8 | 75.0 | 69.7 | 67.5 | 73.7 | 74.9 |
| Alkaline alumina | 84.8 | 85.4 | 91.6 | 86.8 | 81.7 | 87.5 | 88.9 |
| NH₂ | 83.8 | 84.3 | 74.5 | 79.8 | 78.4 | 80.6 | 76.3 |
| GCB | 0.3 | 0.4 | 0.1 | 0.3 | 0.2 | 0.1 | 0.2 |

From the above results, it can be known that QuEChERS method suffers from the problem of analyte loss when using florisil, acidic alumina and GCB. At the same time, PSA, alkaline diatomite and alkaline alumina showed good applicability (above 80%) to most drugs in extractant; on the other hand, although C₁₈, EMR and NH₂ show good removal effects on interferences, excessive use will cause non-negligible losses (<80%). In this context, the dosage of these three absorbents need further optimization.

2. Screening Design

Screening experiment, namely, Plackett-Burman design, can be initially applied to make sure that the factors being further optimized do indeed significantly contribute to the responses and thus narrowing the investigation range of candidate variables. In this respect, Plackett-Burman design is an efficient way to explore multiple factors and screen out the significant ones without concerns about interacting and non-linear effects. As a result, a Plackett-Burman design considering six suspected factors is implemented. Meanwhile, three fictitious factors had also been introduced to determine whether there is systematic error or unknown variable affecting the results. A total of 20 runs with two investigation levels for each factor are involved in the screening design, the matrix is as shown in Table 4.

TABLE 4
The Variables, Coded levels of Plackett-Burman Design

Coded level

| No. | Variables | Level −1 | Level +1 |
|---|---|---|---|
| X₁ | C₁₈ (mg) | 50 | 100 |
| X₂ | PSA (mg) | 50 | 100 |
| X₃ | Fictitious variable-1 | / | / |
| X₄ | EMR (mg) | 50 | 100 |
| X₅ | Alkaline diatomite (mg) | 50 | 100 |
| X₆ | Fictitious variable-2 | / | / |
| X₇ | Alkaline alumina (mg) | 50 | 100 |
| X₈ | NH₂ (mg) | 50 | 100 |
| X₉ | Fictitious variable-3 | / | / |

| Run | X₁ | X₂ | X₃ | X₄ | X₅ | X₆ | X₇ | X₈ | X₉ |
|---|---|---|---|---|---|---|---|---|---|
| 1 | 100 | 100 | / | 50 | 50 | / | 100 | 50 | / |
| 2 | 100 | 50 | / | 50 | 50 | / | 50 | 100 | / |
| 3 | 100 | 100 | / | 50 | 50 | / | 100 | 50 | / |
| 4 | 50 | 100 | / | 50 | 50 | / | 50 | 100 | / |
| 5 | 50 | 100 | / | 50 | 100 | / | 50 | 50 | / |
| 6 | 100 | 100 | / | 100 | 50 | / | 100 | 100 | / |
| 7 | 50 | 50 | / | 100 | 50 | / | 50 | 100 | / |
| 8 | 50 | 50 | / | 50 | 100 | / | 100 | 50 | / |
| 9 | 100 | 100 | / | 100 | 100 | / | 50 | 50 | / |
| 10 | 100 | 50 | / | 100 | 50 | / | 50 | 50 | / |
| 11 | 50 | 50 | / | 50 | 50 | / | 50 | 50 | / |
| 12 | 100 | 50 | / | 50 | 100 | / | 100 | 100 | / |
| 13 | 100 | 100 | / | 50 | 100 | / | 50 | 100 | / |
| 14 | 100 | 50 | / | 100 | 100 | / | 100 | 100 | / |
| 15 | 100 | 50 | / | 100 | 100 | / | 50 | 50 | / |
| 16 | 50 | 100 | / | 100 | 100 | / | 50 | 100 | / |
| 17 | 50 | 100 | / | 100 | 100 | / | 100 | 50 | / |
| 18 | 50 | 100 | / | 100 | 50 | / | 100 | 100 | / |
| 19 | 50 | 50 | / | 100 | 50 | / | 100 | 50 | / |
| 20 | 50 | 50 | / | 50 | 100 | / | 100 | 100 | / |

The critical factors are determined by variance analysis, for instance, the extraction efficiency of acetylfentanyl is affected by X₁, X₄ and X₈ in a significant way, and the order of importance is as follows: X₈>X₁>X₄. Detailed results of the remaining drugs are as follows: isobutyrylfentanyl (X₈>X₁>X₄), acrolylfentanyl (X₈>X₁), ocfentanyl (X₈>X₁), valerylfentanyl (X₈>X₁>X₄), furanylfentanyl (X₁>X₈>X₄), fentanyl (X₁>X₈>X₄). On the other hand, the rest two factors and all the fictitious ones did not exhibit significant effects on the extraction efficiencies in the studied range and are thus set at fixed levels. As a result, the above three critical factors are selected and subjected to further optimization with the aid of response surface methodology-central composite design (CCD).

3. Central Composite Design

After the critical factors had been determined, CCD based on response surface method is designed to investigate the influence of these factors on multiple responses. In a rotatable matrix of CCD (presented in Table 5), each factor is studied at five levels (±α, ±1, 0) to reduce the uncontrollable influences. The numerical values of α depend on the number of experimental factors investigated, and for three factors, it is assigned to 1.68.

TABLE 5
The main factors, symbols, levels and designed matrix of CCD

| Experimental | | Test level | | | | |
|---|---|---|---|---|---|---|
| factor | Symbol | −α | −1 | 0 | 1 | +α |
| C₁₈ dosage (mg) | A | 15.9 | 50.0 | 100.0 | 150.0 | 184.1 |
| EMR dosage (mg) | C | 15.9 | 50.0 | 100.0 | 150.0 | 184.1 |
| NH₂ dosage (mg) | D | 15.9 | 50.0 | 100.0 | 150.0 | 184.1 |

| Run | A | B | C |
|---|---|---|---|
| 1 | 50.0 | 50.0 | 50.0 |
| 2 | 100.0 | 100.0 | 100.0 |
| 3 | 15.9 | 100.0 | 100.0 |
| 4 | 100.0 | 100.0 | 100.0 |
| 5 | 184.1 | 100.0 | 100.0 |
| 6 | 100.0 | 100.0 | 100.0 |
| 7 | 100.0. | 100.0 | 184.1 |
| 8 | 1500 | 50.0 | 150.0 |
| 9 | 50.0 | 150.0 | 150.0 |
| 10 | 100.0 | 15.9 | 100.0 |
| 11 | 100.0 | 100.0 | 100.0 |
| 12 | 100.0 | 100.0 | 100.0 |
| 13 | 150.0 | 150.0 | 50.0 |
| 14 | 100.0 | 100.0 | 15.9 |
| 15 | 50.0 | 150.0 | 50.0 |
| 16 | 50.0 | 50.0 | 150.0 |
| 17 | 100.0 | 184.1 | 100.0 |
| 18 | 100.0 | 100.0 | 100.0 |
| 19 | 150.0 | 50.0 | 50.0 |
| 20 | 150.0 | 150.0 | 150.0 |
| 21 | 50.0 | 50.0 | 50.0 |
| 22 | 100.0 | 100.0 | 100.0 |
| 23 | 15.9 | 100.0 | 100.0 |
| 24 | 100.0 | 100.0 | 100.0 |
| 25 | 184.1 | 100.0 | 100.0 |
| 26 | 100.0 | 100.0 | 100.0 |
| 27 | 100.0 | 100.0 | 184.1 |
| 28 | 150.0 | 50.0 | 150.0 |
| 29 | 50.0 | 150.0 | 150.0 |
| 30 | 100.0 | 15.9 | 100.0 |

The results of each analyte obtained from the designed matrix are fitted to a polynomial equation with quadratic multiple regressions, which express the relationship between response and variable as follow:

Y=δ₀+Σᵢ₌₁^{f}δᵢXᵢ+Σᵢ₌₁^{f}δᵢᵢXᵢ²+Σᵢ₌₁^{f}Σⱼ₌₁^{f}δᵢⱼXᵢXⱼ+ε;

where Y represents the predicted response, Xᵢ and Xⱼ are independent variables, δ₀ is the compensation term while ε is the experimental error. The coefficients, δᵢ, δᵢᵢ and δᵢⱼ represent the linear, interaction and quadratic item, respectively. After deriving the formula from the experimental data, analysis of variance (ANOVA) is employed to identify whether the variations of responses are interpreted by pretreatment experiments or by random errors (results shown in Table 6).

TABLE 6
The Results of ANOVA on the Quadratic Polynomial Models

| Item | Acetylfentanyl | Isobutyrylfentanyl | Acrylylfentanyl | Ocfentanyl | Valerylfentanyl | Furanylfentanyl | Fentanyl |
|---|---|---|---|---|---|---|---|
| Model summary | 0.0001 | 0.0003 | <0.0001 | <0.0001 | <0.0001 | <0.0001 | <0.0001 |
| statistics | | | | | | | |
| Lack of Fit | 0.6544 | 0.2184 | 0.4438 | 0.0836 | 0.4571 | 0.6888 | 0.1615 |
| R² | 0.9300 | 0.9121 | 0.9599 | 0.9623 | 0.9768 | 0.9582 | 0.9377 |
| Adjusted-R² | 0.8670 | 0.8330 | 0.9237 | 0.9283 | 0.9559 | 0.9205 | 0.8816 |
| A-C18 | 0.1153 | 0.0004 | 0.0002 | <0.0001 | <0.0001 | 0.0006 | <0.0001 |
| B-EMR | <0.0001 | 0.0016 | <0.0001 | <0.0001 | <0.0001 | <0.0001 | <0.0001 |
| C-NH₂ | 0.0019 | <0.0001 | <0.0001 | <0.0001 | <0.0001 | <0.0001 | <0.0001 |
| AB | 0.4453 | 0.1810 | 0.7637 | 0.6080 | 0.0372 | 0.3404 | 0.1449 |
| AC | 0.7572 | 0.2667 | 0.0648 | 0.6892 | 0.0871 | 0.4833 | 0.5057 |
| BC | 0.8769 | 0.8319 | 0.4703 | 0.0028 | 0.1000 | 1.0000 | 0.8792 |
| A² | 0.0301 | 0.4124 | 0.3867 | 0.5548 | 0.2698 | 0.7158 | 0.0545 |
| B² | 0.0122 | 0.1010 | 0.0100 | 0.2670 | 0.4161 | 0.0125 | 0.3244 |
| C² | 0.5359 | 0.0598 | 0.2947 | 0.2291 | 0.0008 | 0.2147 | 0.1757 |

Model summary statistics with a maximum P-value of 0.0003 indicated that all the generated models are significant, and the variations of responses could be explained by the polynomial models, rather than with the pure error (<0.01%). The “Lack of fit” P-value (from 0.0836 to 0.6888) indicated that the polynomial models fitted the data well and are not aliased for further analysis. The coefficient of determination (R²) is determined using the least square regression and applied to evaluate the overall variation in the data accounted by the model, and the R² should be at least 0.800 to verify the favorable consistency between the actual data and theoretical predictions. As a result, the R² (minimum 0.9121) and the Adjusted-R² (minimum 0.8330) implied that the established polynomial models have a 91.21% agreement with the experimental data and can explain the variation effect of 83.30%. On the other hand, the significances of independent variables and interaction effects are determined by the student's t-test. For instance, the independent variable of B and C, and quadratic term of A², B² exhibited significant effects on the results of acetylfentanyl. Finally, numerical optimization is carried out by setting the goals for three variables (in range of 0 mg to 200 mg) and each response (maximum). As a result, the experimental combination of three sorbents (27 mg of C₁₈, 29 mg of EMR, 143 mg of NH₂) with the highest desirability is chosen (predicted recoveries are predicted to be greater than 85%). At the same time, Considering the purification effects of PSA, alkaline diatomite and alkaline alumina on the metal ions, pigments, organic acids and phenolic interferences, these adsorbents are still reserved for usage. In summary, the proportion of the mixed purification adsorbents is set as follows: 27 mg of C₁₈, 29 mg of EMR, 143 mg of NH₂, and 100 mg of PSA, alkaline diatomite and alkaline alumina.

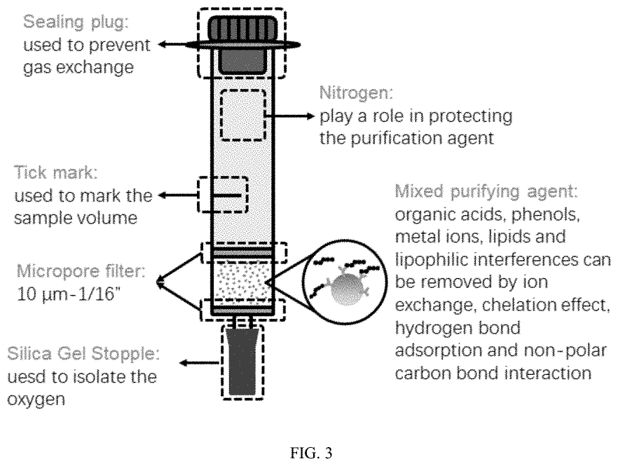

After the proportion and dosage of the mixed adsorbents had been determined, they are filled into blank solid-phase column and fixed using two 10-μm sieve plates. The bottom end of the unused purification tube is sealed with a silica gel plug, and the top end is blocked with a plastic seal plug. Nitrogen is employed as shielding gas to reduce the reaction between oxygen and the sorbents. There is a scale line on the surface of the purification tube with the aim of indicating the volume of sample solution required. When the sample solution reaches the scale line, the bottom of the purification tube is removed from the liquid surface and a certain amount of air (>1 mL) continues to be sucked in. After that, the plunger is used to push out the sample solution to make secondary contact with the sorbents, meanwhile, the air helps to drain as much liquid as possible. The design diagram of the purification extraction tube assembly is shown in FIG. 3.

4. Validation of the Established Method

Analytical characteristics of the proposed method are evaluated by a validation procedure with spiked biological samples, in terms of purification effect, linearity, matrix effect (ME), accuracy, repeatability, inter-day precision, limits of quantification (LOQs) and selectivity.

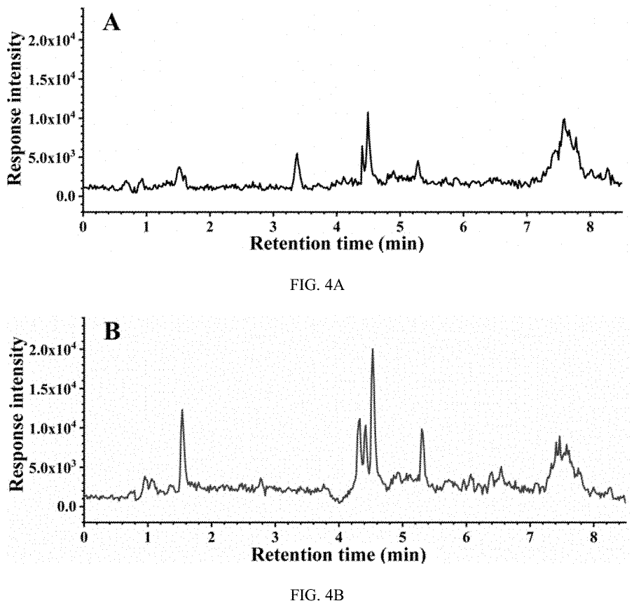

Firstly, the purification effect of the designed tube is investigated in the plasma matrix: (1) when the purification extraction tube assembly is used, the yellow components in the plasma will be significantly adsorbed and as a result, no obvious pigment components are visible to the naked eye in the final sample solution; (2) the difference of purification effect is reflected in the form of mass spectrometry total ion chromatogram (see FIGS. 4A and 4B), and (3) FIG. 4A shows the signal of purified extractant; (4) FIG. 4B represents that the direct injection of sample solution without purification. It can be observed that the baseline of the total ion chromatogram is smoother after clean-up using the purification extraction tube assembly. Meanwhile, the signals of interferences around the retention time of 1.5 min and 4-5 min are also lower, and moreover, the latter period coincided with the retention time of three fentanyl drugs (isobutyrylfentanyl, valerylfentanyl and furanylfentanyl).

Because of ionization interrupting in ESI source by co-elution matrix components, matrix effects are choosing to evaluate the effect to target analytes. Although the isotopic internal standards are employed to correct the signal suppression or enhancement effects, matrix effect is still estimated as a criterion for the assessment of purification effects of the extractant. In this case, the signal suppression or enhancement effects are determined by comparing the slope of matrix matched curve versus the slope of pure standard curve. Both these two curves are freshly constructed at ten concentration levels each batch: 0.05 ng/mL, 0.10 ng/mL, 0.20 ng/mL, 0.50 ng/mL, 1.0 ng/mL, 2.0 ng/mL, 5.0 ng/mL, 10.0 ng/mL, 20.0 ng/mL and 50.0 ng/mL. The matrix effects are generally considered tolerable within ±20%, and the results are summarized in Table 7. As shown, the matrix effects of all fentanyl drugs in the three investigated matrices are not significant (81.5%-108%), which indicated that the purification effect of the proposed method is satisfactory.

TABLE 7
Results of Linearity and Matrix Effects in Three Investigated Matrices

| Target | Parameters | Standard solution | Blood plasma | Saliva | Urine |
|---|---|---|---|---|---|
| Acetylfentanyl | Linear | y = 8.02 × 10⁵x + | y = 6.84 × 10⁵x + | y = 8.36 × 10⁵x − | y = 8.01 × 10⁵x + |
| | equation | 4.79 × 10⁴ | 2.35 × 10⁴ | 4.67 × 10³ | 5.34 × 10⁴ |
| | Matrix | | 85.3% | 104% | 99.8% |
| | effect | | | | |
| Isobutyrylfentanyl | Linear | y = 7.29 × 10⁵x + | y = 7.25 × 10⁵x + | y = 7.09 × 10⁵x + | y = 7.38 × 10⁵x + |
| | equation | 1.03 × 10⁵ | 3.06 × 10⁴ | 1.60 × 10⁵ | 1.19 × 10⁵ |
| | Matrix | | 99.4% | 97.2% | 101% |
| | effect | | | | |
| Acroloylfentanyl | Linear | y = 6.64 × 10⁵x + | y = 5.98 × 10⁵x + | y = 6.50 × 10⁵x + | y = 6.62 × 10⁵x + |
| | equation | 5.71 × 10⁴ | 1.24 × 10⁴ | 8.59 × 10⁴ | 7.08 × 10⁴ |
| | Matrix | | 90.1% | 97.9% | 99.7% |
| | effect | | | | |
| Ocfentanyl | Linear | y = 6.29 × 10⁵x + | y = 6.83 × 10⁵x − | y = 6.17 × 10⁵x + | y = 6.32 × 10⁵x + |
| | equation | 7.45 × 10⁴ | 4.88 × 10⁴ | 9.41 × 10⁴ | 7.98 × 10⁴ |
| | Matrix | | 108% | 98.1% | 100% |
| | effect | | | | |
| Valerylfentanyl | Linear | y = 8.61 × 10⁵x + | y = 8.17 × 10⁵x − | y = 8.26 × 10⁵x + | y = 8.09 × 10⁵x + |
| | equation | 1.24 × 10⁵ | 1.75 × 10⁴ | 1.26 × 10⁵ | 1.02 × 10⁵ |
| | Matrix | | 94.9% | 95.9% | 94.0% |
| | effect | | | | |
| Furanylfentanyl | Linear | y = 9.94 × 10⁵x + | y = 9.12 × 10⁵x − | y = 8.27 × 10⁵x + | y = 8.10 × 10⁵x + |
| | equation | 1.47 × 10⁵ | 2.62 × 10⁴ | 1.26 × 10⁵ | 1.02 × 10⁵ |
| | Matrix | | 91.7% | 83.2% | 81.5% |
| | effect | | | | |
| Fentanyl | Linear | y = 2.81 × 10⁵x + | y = 2.47 × 10⁵x − | y = 2.72 × 10⁵x + | y = 2.81 × 10⁵x + |
| | equation | 7.05 × 10³ | 1.16 × 10³ | 1.82 × 10⁴ | 9.91 × 10³ |
| | Matrix | | 87.9% | 96.8% | 100% |
| | effect | | | | |

Note:
Satisfactory linear relationships with R² higher than 0.999 in all the case are achieved.

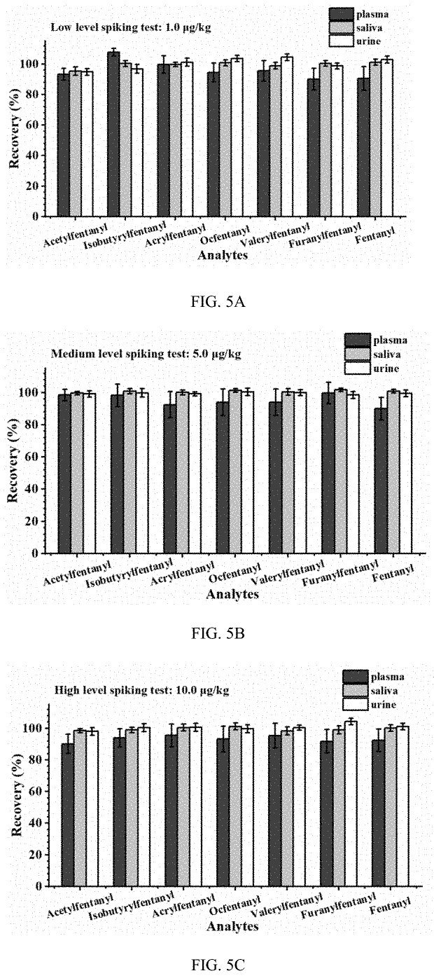

The accuracy of the proposed method is assessed through the three-level spiking tests (n=6, 1.0 μg/kg, 5.0 μg/kg and 10.0 μg/kg, respectively) in three matrices while the precision is expressed in terms of RSDs (see FIGS. 5A-5C). As results shown, the recovery rates of fentanyl drugs are above 90% in all tested assays, with the associated RSDs not exceeding 8.2%. Then, the sensitivity of the proposed method in two matrices are estimated by spiking various low concentration levels and determined as the lowest concentrations producing signal-to-noise ratio (S/N) of 3 and 10, respectively. Finally, the limit of detection (LOD) and quantification (LOQ) of fentanyl is 0.2 μg/kg and 0.5 μg/kg, respectively, meanwhile, the LOD and LOQ for the other six fentanyl drugs is 0.1 μg/kg and 0.3 μg/kg, respectively. According to the above data, the analytical performance of this methodology, including the scope of application, purification capacity, accuracy and precision, are comparable to or better than the previously reported methods, and can completely meet the purpose of rapid analysis of fentanyl concentration in body fluid samples.

The above-described embodiments merely describe the preferred embodiments of the disclosure and are not intended to limit the scope of the disclosure, and various modifications and changes thereof made by those of ordinary skilled in the art without departing from the design spirit of the disclosure shall fall within the scope of protection determined by the appended claims of the disclosure.

What is claimed is:

1. A method for determination of a fentanyl drug in a biological sample, using liquid chromatography tandem mass spectrometry (LC-MS/MS) for determining a content of the fentanyl drug in the biological sample, and specifically comprising:
step (1) sample pretreatment: shaking and centrifuging the sample sequentially, and then removing co-extraction impurities in an extract of the centrifuged sample by a purification extraction tube assembly for fentanyl drugs to thereby obtain a target solution;
step (2) LC-MS/MS based analysis on the target solution: using 0.1% by volume of formic acid-aqueous solution and 0.1% by volume of formic acid-acetonitrile as mobile phases for liquid chromatography analysis, and using a multiple-reaction monitoring (MRM) mode under positive c (ESI) for mass spectrometry analysis.

2. The method according to claim 1, wherein the fentanyl drug comprises one selected from the group consisting of: acetylfentanyl, isobutyrylfentanyl, acrylfentanyl, ocfentanyl, fentanyl, valerylfentanyl and furanylfentanyl.

3. The method according to claim 1, wherein the purification extraction tube assembly in the step (1) comprises: mixed purifying agents, a solid phase extraction column, a sieve plate, a syringe plunger, and a filter membrane.

4. The method according to claim 1, wherein the sample comprises one selected from the group consisting of whole blood, saliva and urine.

5. The method according to claim 3, wherein components and dosages of the mixed purifying agents are as follows: 27 milligrams (mg) of cyclo[18]carbon (C₁₈), 29 mg of EMR (Bond Elut EMR-Lipid), 143 mg of NH₂ (aminopropyl), 100 mg of PSA (primary secondary amine), 100 mg of alkaline diatomite and 100 mg of basic alumina.

6. The method according to claim 5, wherein the components and dosages of the mixed purifying agents are determined by chemometrics, and the chemometrics comprises Plackett-Burman design and central composite design based on response surface methodology.

7. The method according to claim 3, wherein the filter membrane is a 0.22 μm hydrophilic PTFE (polytetrafluoroethylene) millipore filtration membrane.

8. The method according to claim 3, wherein the shaking and centrifuging the sample sequentially in the step (1) comprise: adding the sample into acetonitrile with a volume twice of a volume of the added sample to obtain a mixture and shaking the mixture for 5 minutes (min), and then centrifuging the mixture at 15000 revolutions per minute (rpm) at 4° C. for 10 min after the shaking;
wherein a supernatant obtained after the centrifuging is aspirated into the purification extraction tube assembly to make the supernatant in full contact with the mixed purifying agents, and then pushed out after installing the filter membrane in the front of the solid phase extraction column.

9. The method according to claim 1, wherein conditions for the liquid chromatography analysis in the step (2) are as follows: mobile phase A: 0.1% formic acid solution (V/V), mobile phase B: 0.1% formic acid-acetonitrile (V/V), chromatography column: Waters BEH (Ethylene-Bridged-Hybrid) C₁₈ column, flow rate: 300 microliters per minute (μL/min), and injection volume: 10 μL; and
wherein conditions for the mass spectrometry analysis are as follows: electrospray ionization in positive mode is performed in multiple-reaction monitoring (MRM) conditions, nitrogen is used as curtain gas and collision gas at 20.0 pounds per square inch (psi) and 7.0 psi respectively, declustering voltage: 120 volts (V), ionspray voltage maintained at 4.5 kilovolts (kV), and source cone temperature: 500° C.

10. A kit for determination of a fentanyl drug in a biological sample according to the method as claimed in claim 1, comprising:
a purification extraction tube assembly, polypropylene centrifuge tubes prefilled with acetonitrile, standard working solutions, quality-control samples, and several disposable consumables.
